(19) United States
(12) Patent Application Publication
(10) Pub. No.: US 2014/0242653 A1
(43) Pub. Date: Aug. 28, 2014

(54) METHOD FOR PRODUCING PYRUVIC ACID FROM ALGINIC ACID

(71) Applicants: MARUHA NICHIRO FOODS, INC., TOKYO (JP); KYOTO UNIVERSITY, KYOTO-SHI (JP)

(72) Inventors: KOUSAKU MURATA, KYOTO-SHI (JP); SHIGEYUKI KAWAI, KYOTO-SHI (JP); NOBUYUKI SATO, TSUKUBA-SHI (JP)

(73) Assignees: MARUHA NICHIRO FOODS, INC., TOKYO (JP); KYOTO UNIVERSITY, KYOTO-SHI (JP)

(21) Appl. No.: 14/189,513

(22) Filed: Feb. 25, 2014

(30) Foreign Application Priority Data
Feb. 25, 2013 (JP) 2013-034710
Aug. 23, 2013 (JP) 2013-173668

(51) Int. Cl. C12P 7/40

(52) U.S. Cl. CPC C12P 7/40
USPC 435/136

(57) ABSTRACT

A method for producing pyruvic acid from polysaccharide alginic acid that is contained in large amounts in brown algae using the ability to assimilate alginic acid possessed by a lactate-dehydrogenase-gene-deficient Sphingomonas sp. A1 strain (ldh strain) is provided. The method for producing pyruvic acid comprises culturing the lactate-dehydrogenase-gene-deficient Sphingomonas sp. A1 strain (ldh strain) in a medium containing alginic acid, and causing the strain to produce pyruvic acid from alginic acid and then to secrete pyruvic acid into the medium.

### BACKGROUND OF THE INVENTION

1. Field of the Invention

The present invention relates to a method for producing pyruvic acid from alginic acid using alginic acid-assimilating bacteria.

2. Background Art

Long-term measures for prevention of global warming and food security throughout the world are important matters, for which global solutions are required. These matters were discussed at the 34^{th} G8 summit meeting in 2008 and a statement was made. Effective utilization of natural energy and infrequently used non-food biomass is essential for realization of the aims of the statement. A report summarizing the importance of the development of such effective utilization has also been submitted (Non-Patent Document 1).

Non-food biomass can be roughly divided into two types of biomass: terrestrial and aquatic/marine. Regarding terrestrial biomass-derived components such as glucose and starch, the fermentative production of ethanol and other useful compounds using these components as substrates has been turned into practical applications. However, studies on and the development of aquatic/marine biomass-derived components have been delayed in comparison. When terrestrial biomass-derived components are used, the problem of competing with foodstuffs and land problems are unavoidable and also unignorable particularly in Japan where the amount of area under cultivation is extremely small.

On the other hand, as a seafaring country, Japan possesses a vast exclusive economic zone and the amount of marine biomass produced in this zone is huge. In particular, large brown algae can be particularly promising biomass because of the rich amount of algal body and the high growth rate.

One of major components of brown algae is the acidic polysaccharide, alginic acid, which accounts for about 30% to 60% in the dry algal body. Examples of the applications of alginic acid include functional materials (Patent Document 1), medical materials, and food materials (Patent Documents 2 and 3) prepared by degrading alginic acid itself into low molecules for purification. However, the application range of these materials is limited and the molecules require further purification or the like before use.

Regarding another application thereof, a method for producing ethanol has been reported as a method for producing biomass energy from alginic acid (Patent Document 4). This technology is epoch-making since biomass energy is generated from alginic acid and is of particular value as a basic technology. However, the building of a technology capable of producing not only ethanol, but also other useful components with the use of the same or similar raw materials and facilities is desired for the establishment of an independent industry from which operating revenue can be anticipated.

However, regarding a method for generating a useful compound by a fermentation method using alginic acid as a raw material, no example of producing a compound other than ethanol has been reported.

Pyruvic acid is an important compound that is broadly used as a raw material for synthesizing various chemical substances and polymers, or as an additive or an ingredient in foods and pharmaceutical products. Currently, fermentation methods using microorganisms such as Escherichia coli, bacteria of a coryneform group, and yeast using glucose as a substrate are known. However, there is no known example of the production of pyruvic acid from alginic acid derived from marine biomass that is a promising carbon source.

A general example of an industrial method for producing pyruvic acid is a method for obtaining pyruvic acid by decarboxylation of tartaric acid. This production method involves the generation of CO₂ in a quantity that is equimolar to the product, has a long-time reaction at a high temperature, and mass disposal of sulfate, and thus causes a large environmental burden and requires a large input of energy. Therefore, a new production method is desired.

As a method for producing pyruvic acid by a fermentation method, methods using glucose as a raw material and microorganisms of the genus Saccharomyces, the genus Candida, and the genus Torulopsis have been reported as described above (e.g., Patent Documents 5-7).

However, the method using glucose is problematic in competing with foodstuffs. Moreover, there are various applications of glucose itself, including an application for foods. Production by such a fermentation method tends to result in rising cost. Raw materials to be desirably used herein are unused or infrequently used resources.

### PRIOR ART DOCUMENTS

### Patent Documents

Patent Document 1: JP Patent Publication (Kokai) No. 2000-313880 A
Patent Document 2: JP Patent No. 3825069
Patent Document 3: JP Patent No. 3505185
Patent Document 4: JP Patent No. 4845070
Patent Document 5: JP Patent Publication (Kokoku) No. 51-34475 B (1976)
Patent Document 6: JP Patent Publication (Kokoku) No. 3-5800 B (1991)
Patent Document 7: JP Patent Publication (Kokoku) No. 3-58275 B (1991)

### Non-Patent Documents

Non-Patent Document 1: Science and Technology Future Strategy Workshop, Effective Use of Natural Energy-Approach from Materials-Report on Base Technology for Biofuel Production Using Microorganisms, Center for Research and Development Strategy, Japan Science and Technology Agency, 2008 November

### SUMMARY OF THE INVENTION

### Object to be Attained by the Invention

An object of the present invention is to provide a method for producing pyruvic acid by a fermentation method using alginic acid as a raw material contained in large amounts in marine biomass, and specifically, not from terrestrial biomass, but from infrequently used marine biomass.

### Means for Attaining the Object

The present invention relates to production of pyruvic acid that can be broadly used as a raw material for synthesizing various chemical substances and polymers and also as an additive for foods and pharmaceutical products, from alginic acid, which is a major component of brown algae, a particularly promising biomass among the infrequently used types of marine biomass. No method for producing pyruvic acid from alginic acid by a fermentation method has ever been disclosed.

Pyruvic acid is an intermediary metabolite of the energy metabolic pathway of a strain. This energy metabolic pathway works with various metabolic pathways and then the product itself serves as a signal for homeostasis. In this manner, metabolic pathways are strictly controlled to avoid the runway processes of various important metabolic pathways, including the energy metabolic pathway. Because of these factors, intermediary metabolites are immediately metabolized to some final metabolites. In general, intermediary metabolites themselves do not remain for long periods of time.

When the preceding energy metabolic pathway is blocked by some measures in order to obtain an intermediary metabolite, the intermediary metabolite is immediately metabolized via another metabolic pathway that works therewith. When a plurality of metabolic pathways that follow the production of a target intermediary metabolite are inappropriately stopped in order to obtain the intermediary metabolite, strains themselves will be unable to survive and die since metabolic pathways required for their survival have been blocked.

Furthermore, when a method for producing some sort of useful substance by a fermentation method is industrialized, a method for efficiently recovering the thus produced target useful substance should be examined. If the target substance is the final metabolite, it is discharged outside the cells in many cases and can be recovered. For example, ethanol is the final metabolite and falls under the case. However, intermediary metabolites in the energy metabolic pathway are used in various metabolic pathways working in conjunction therewith, and thus are rarely discharged from the cells. In order to recover a target substance in such a case, a strain containing the thus produced target substance is collected and then disrupted to recover the substance.

This means that serial cultures cannot be performed. Such procedures have very poor productivity, and require disruption of the strains every time the target substance is recovered and separation and purification of the target substance. Stable growth of a strain often requires a culture period, and stabilization of production is extremely difficult. Whether or not stable production can be achieved is a major factor in the industrialization of a process. Even if an unused or infrequently used resource can be used as a raw material, industrialization is impossible unless production itself can be continuously performed.

As a result of intensive studies, the present inventors have discovered that pyruvic acid, which is an intermediary metabolite of an energy metabolic pathway, can be efficiently discharged outside the cells without killing the microorganism by disrupting the lactate dehydrogenase gene of Sphingomonas sp. A1 strain, which is capable of assimilating alginic acid, and then culturing the strain under high-oxygen conditions. As a result, the present inventors have discovered that: pyruvic acid is produced from alginic acid by a fermentation method through serial culture of the microorganism under the aforementioned conditions; and pyruvic acid is continuously produced from alginic acid by a fermentation method through serial culture of a strain that discharges the produced pyruvic acid outside the strain. Through serial culture of these microorganisms over a period of time, pyruvic acid can be continuously produced in an efficient and safe manner from alginic acid by a fermentation method.

Specifically, the present invention is as follows.

[1] A method for producing pyruvic acid, comprising culturing a lactate-dehydrogenase-gene-deficient Sphingomonas sp. A1 strain (ldh strain) in a medium containing alginic acid and causing the strain to produce pyruvic acid from alginic acid and then to secrete pyruvic acid into the medium.
[2] The method for producing pyruvic acid of [1], wherein the concentration of alginic acid at the initiation of culture ranges from 2 to 10 (w/v) % and the biomass of the lactate-dehydrogenase-gene-deficient Sphingomonas sp. A1 strain (ldh strain) at the initiation of culture is represented by a value for A₆₀₀ ranging from 0.075 to 1.25 in terms of turbidity.
[3] The method for producing pyruvic acid of [1] or [2], wherein culture is performed under conditions in which the maximum dissolved oxygen concentration in a medium is 15% or more.
[4] The method for producing pyruvic acid of any one of [1] to [3], wherein culture is performed for 2 to 3 days.
[5] The method for producing pyruvic acid of any one of [1] to [4], wherein culture is performed by shake culture at a shaking frequency of 100 spm (strokes per minutes) or higher.
[6] The method for producing pyruvic acid of any one of [1] to [5], wherein culture is performed at pH 6.0 to 8.0.
[7] The method for producing pyruvic acid of any one of [1] to [6], wherein the concentration of pyruvic acid secreted into the medium on day 2 of culture is 2.5 g/l or greater.
[8] The method for producing pyruvic acid of any one of [1] to [6], wherein the concentration of pyruvic acid secreted into the medium on days 2 to 3 of culture is 5 g/l or greater.

### Effects of the Invention

The present invention makes it possible to produce pyruvic acid from polysaccharide alginic acid that is contained in large amounts in marine-derived biomass and particularly in brown algae. Therefore, the development of a novel method for producing pyruvic acid is achieved not using a raw material (cellulosic or farinaceous) mainly consisting of glucose that always competes with foodstuffs, but using as a raw material biomass that does not compete with foodstuffs, can be re-produced in a marine area, and is mainly composed of uronic acid. Thus, a significant social effect can be expected.

Moreover, a novel method for producing pyruvic acid not involving the generation of CO₂ in a quantity that is equimolar to the product, a long-time reaction at a high temperature, a large environmental burden such as mass disposal of sulfate, and a large input of energy consumption can be provided.

Furthermore, the method that can be provided herein comprises: efficiently blocking an energy metabolic pathway without killing microorganisms, which has been thought to be difficult; and causing the microorganisms to discharge a target intermediary metabolite outside the cells.

The description includes the contents described in the description and/or drawings of JP Patent Application Nos. 2013-034710 and 2013-173668 based on which the priority of the present application is claimed.

### BRIEF DESCRIPTION OF THE DRAWINGS

FIG. 1 shows an ethanol production pathway of the ethanol-producing Sphingomonas sp. A1 ldh strain (MK3353 strain) for producing ethanol from alginic acid (reproduced from FIG. 1 of Takeda et al. Energy Environ. Sci. 4: 2575-2581 (2011)).

FIG. 2 shows the effects of the amount of pyruvic acid in a medium on the growth of the Sphingomonas sp. A1 WT strain and ldh strain. FIG. 2 shows the growth of each strain when it was cultured for 24 hours in a 0.8% (w/v) alginic acid medium containing pyruvic acid (at each concentration).

FIG. 3 shows the effects of the shaking frequencies of shake culture on: dissolved oxygen concentration in a medium; and the pyruvic acid production (amount of pyruvic acid produced), the alginic acid consumption (amount of alginic acid consumed), and the growth of the control Sphingomonas sp. A1 ldh strain (MK3567 strain).

FIG. 4 shows HPLC analyses of differences in produced compounds resulting from shaking frequencies of shake culture. The supernatants of culture solutions on day 4 of culture in FIG. 3 were analyzed by HPLC.

FIG. 5 shows the effects of the initial concentrations of alginic acid on the pyruvic acid production, the alginic acid consumption, and the growth of the Sphingomonas sp. A1 ldh strain, as well as alginic acid consumption by the strain.

FIG. 6 shows the effects of lactate dehydrogenase (LDH) gene deficiency on the pyruvic acid production, the alginic acid consumption, the lactic acid production, and the growth of strains.

WT; Sphingomonas sp. A1 WT strain.
ldh; Sphingomonas sp. A1 ldh strain.

FIG. 7-1 shows the growth of the Sphingomonas sp. A1 ldh strain when the initiation alginic acid concentration ranged from 5% to 10%.

FIG. 7-2 shows the pyruvic acid production of the Sphingomonas sp. A1 ldh strain when the initiation alginic acid concentration ranged from 2% to 10%.

FIG. 8 shows the effects of pHs in culture solutions on the pyruvic acid production of the MK2651 strain (the Sphingomonas sp. A1 ldh strain).

FIG. 9 shows the results of culturing the MK2651 strain (the Sphingomonas sp. A1 ldh strain) when the pHs of culture solutions were maintained at 6.5 and 7.5.

### DESCRIPTION OF THE PREFERRED EMBODIMENTS

Hereafter, the present invention is described in detail.

In the present invention, the lactate dehydrogenase (LDH) gene of the Sphingomonas sp. A1 strain is disrupted, alginic acid is administered as a raw material, and then the strain is continuously cultured under high-oxygen conditions, and thus pyruvic acid is produced continuously and stably by a fermentation method from the polysaccharide alginic acid of brown algae, which is an infrequently used marine biomass, without causing a high degree of environmental burden.

The Sphingomonas sp. A1 strain has been isolated by Dr. Kosaku Murata et al., Graduate School of Agriculture, Kyoto University, as described in Hisano, T. et al, Biochem. Biophys. Res. Commun., 220, 979-982. The Sphingomonas sp. A1 strain exhibits extremely strict auxotrophy and specifically uses uronic-acid-containing polysaccharides such as alginic acid and pectin as carbon sources (Hisano, T. et al, J. Ferment. Bioeng., 79, 538-544 (1995)).

The Sphingomonas sp. A1 strain may be a strain to which resistance to pyruvic acid has been imparted (made resistant to pyruvic acid or tolerized to pyruvic acid). For example, a pyruvic acid-resistant strain can be obtained by conditioning and culturing a strain in a medium containing pyruvic acid, and then inducing a random mutation by ultraviolet irradiation. In the present invention, a thus isolated pyruvic acid-resistant strain may be used. Also, a lactate-dehydrogenase-deficient Sphingomonas sp. A1 strain (ldh strain) that has not been made resistant to pyruvic acid is resistant to pyruvic acid concentrations to some degree, such as 100 mM and preferably 150 mM.

The Sphingomonas sp. A1 strain is capable of assimilating alginic acid, such that the strain forms cavities on cell surface layers in the presence of alginic acid, incorporates alginic acid as it is (in the form of a polysaccharide) into cells, and then degrades it. Specifically, the Sphingomonas sp. A1 strain incorporates polysaccharide alginic acid into cells via cavities and an ABC transporter, and then degrades it into monosaccharides using endo/exo-type alginate lyase. A monosaccharide is non-enzymatically cleaved to result in keto acid (DEH; 4-deoxy-L-erythro-5-hexoseulose uronic acid), keto acid is converted to pyruvic acid and glyceraldehyde-3-phosphate (G-3-P) by a keto acid metabolic enzyme group, and then the resultants are immediately used for energy production in the TCA cycle. Only some thereof are converted into ethanol and then released into the medium. Moreover, some of pyruvic acid and G-3-P are converted to ethanol.

For partially and completely blocking of the lactate dehydrogenase of the Sphingomonas sp. A1 strain, genes encoding enzymes involved in the synthesis of these substances are knocked out. Gene knockout can be performed by a known method, such as homologous recombination.

Furthermore, a metabolic pathway following the conversion to pyruvic acid may be changed by blocking it. For example, when the pyruvate carboxylase reaction pathway is partially or completely blocked, pyruvic acid is produced and accumulated at even higher levels without being converted to oxalacetic acid. For partial or complete blocking of the pyruvate carboxylase reaction pathway, a pyruvate carboxylase gene is knocked out. Gene knockout can be performed by a known method, such as homologous recombination.

Furthermore, an example of a method for producing pyruvic acid using the ability of the Sphingomonas sp. A1 strain to assimilate alginic acid and polysaccharide alginic acid as a raw material is a method that involves introducing genes encoding proteins and enzymes associated with the ability of the Sphingomonas sp. A 1 strain to assimilate alginic acid into another microorganism, causing the microorganism to incorporate alginic acid, and then causing it to produce pyruvic acid using alginic acid as a raw material within the microbial body. Examples of a microorganism include a microorganism of the genus Escherichia coli (E. coli), a microorganism of the genus Sphingomonas, a microorganism of the genus Pseudomonas, a microorganism of the genus Bacillus, and a microorganism of the genus Corynebacterium. Examples of proteins and enzymes associated with the ability to assimilate alginic acid include proteins involved in incorporation of alginic acid into microbial body, enzymes for degrading alginic acid to generate a-keto acid, and keto acid-metabolizing enzymes that form pyruvic acid from keto acid. Examples of proteins involved in incorporation of alginic acid into microbial body include an ABC transporter and an alginic acid-binding protein.

Examples of an enzyme include endo-type/exo-type alginate lyase. As keto acid-metabolizing enzymes, NADH-dependent a-keto acid reductase (A1-R), kinase (A1-K), and aldolase (A1-A) can be used.

A gene group involved in incorporation of alginic acid and a gene group involved in degradation of alginic acid form clusters in the genome of the Sphingomonas sp. A1 strain. The ABC transporter forms a heterotetramer (A1gM1-A1gM2/A1gS-A1gS). The nucleotide sequences of the genes encoding A1gM1, A1gM2, and A1gS are shown in SEQ ID NOS: 1, 2 and 3, respectively. In the alginic acid-binding protein, A1gQ1 and AlgQ2 are present. The nucleotide sequences of the genes encoding A1gQ1 and AlgQ2 are shown in SEQ ID NOS: 4 and 5, respectively. The endo-type alginate lyase (Aly) contains 3 molecular species (A1-I, A1-II and A1-III) within the molecule. The nucleotide sequence of the gene encoding endo-type alginate lyase (Aly) is shown in SEQ ID NO: 6. The nucleotide sequence of the gene encoding exo-type alginate lyase (A1-IV) is shown in SEQ ID NO: 7. The nucleotide sequence of the gene encoding keto acid reductase (A1-R) is shown in SEQ ID NO: 8.

These genes can be introduced using amphotropic vectors (IncP, IncQ, IncW-type plasmids). Examples of an amphotropic vector include plasmid DNA and phage DNA (e.g., pKS13, pJRD215, and pUFR027). These genes may be separately inserted into different vectors, or a plurality of genes may be inserted into one vector. When a plurality of vectors are used, vectors are selected with attention to incompatibility among vectors. A method for introducing a recombination vector into a microorganism is not particularly limited as long as it is a method for introducing DNA into a microorganism. Examples thereof include a method using a calcium ion [Cohen, S. N. et al.: Proc. Natl. Acad. Sci., U.S.A., 69: 2110 (1972)], electroporation, and tri-parental mating.

Microorganisms are cultured according to a general method that is employed for culturing a host, wherein alginic acid is added to a known medium. As alginic acid to be used as a raw material, alginates such as sodium alginate, potassium alginate, calcium alginate, and ammonium alginate, and alginate oligosaccharide can be used. When sodium alginate is used, it is added at a concentration ranging from 2 to 10 (w/v) %, preferably 3 to 8 (w/v) %, and further preferably 4 to 7 (w/v) %. When alginate oligosaccharide is used, it is added at a concentration ranging from 5 to 15 (w/v) %. When the concentration of sodium alginate at the time of initiation of culture ranges from 4 to 5 (w/v) %, a microorganism grows most favorably and exhibits the highest pyruvic acid concentration (pyruvic acid production). The initial alginic acid concentration ranging from 3 to 8 (w/v) % has a critical effect on the production of pyruvic acid. Furthermore, alginic acid may be fed over time. The quantity of alginic acid can be determined by the method described in Knutson, C. A. et al., (1968) Anal. Biochem., 24, 470-481, for example.

Culture is performed under aerobic conditions such as aeration culture with shaking or aeration-agitation culture. The term “shake culture or culture with shaking” refers to a culture method that involves mixing cells with a medium by shaking the culture container. The term “aeration-agitation culture” refers to a culture method that involves mixing cells with a medium using a stirrer, agitation blades or the like while aeration. Particularly when pyruvic acid is produced by the method of the present invention, oxygen should be sufficiently supplied. Thus, shake culture is preferably performed at a high shaking frequency (spm: strokes per min). For example, shake culture is performed at 100 spm or more, preferably 120 spm or more, further preferably 130 spm or more, and particularly preferably 140 spm or more, such as 145 spm. Furthermore, culture may also be performed under conditions where the oxygen partial pressure is increased. Alternatively, culture may also be performed under a high dissolved oxygen condition with aeration of an oxygen gas. Oxygen is dissolved in a medium by the above culture method. For example, in shake cultures at 100 spm or more, preferably 120 spm or more, further preferably 130 spm or more, and particularly preferably 140 spm or more, such as 145 spm, the dissolved oxygen level in the medium after the initiation of culture decreases once. However, after more than ten hours of culture, the dissolved oxygen level increases, reaches about 20% within 2 to 3 days, and then reaches saturation. Meanwhile, in shake cultures at less than 100 spm, such as 95 spm or 50 spm, the dissolved oxygen concentration will never increase during culture. Furthermore, oxygen is dissolved in advance in a medium, and then culture may be performed with a dissolved oxygen concentration of 5% or more, preferably 10% or more, and further preferably 20% or more. Culture may be performed under culture conditions wherein at least the maximum dissolved oxygen concentration is 10% or more, and preferably 15% or more during culture. Culture may be preferably performed under conditions where preferably after 2 days of culture, the dissolved oxygen concentration is as specified above in the medium. For example, shake culture is performed at the above shaking frequency. The culture temperature ranges from 20° C. to 40° C., and preferably ranges from 28° C. to 32° C. The pH ranges from pH6.0 to 9.0, preferably ranges from pH6.0 to 8.0, further preferably ranges from pH6.5 to 7.5, further preferably ranges from pH6.5 to 7.2, further preferably ranges from pH6.5 to 7.0, and is particularly preferably pH7.0. Regarding the culture period, culture is performed for several hours to several days, such as 2 to 4 days, preferably 2 to 3 days, and further preferably 2 days. A large amount of pyruvic acid is produced during 1 to 3 days of culture, and the assimilation of pyruvic acid is begun on and after around day 4 of culture and thus the amount thereof decreases. Hence, long-term culture is not preferable. The pH of a medium is adjusted using inorganic or organic acid, an alkaline solution, or the like. Specifically, the pH is adjusted using HCl or NaOH. The pH may be adjusted periodically during the culture period, such as every 12 to 24 hours, and preferably every 24 hours. Alternatively, the pH may be adjusted continuously during the culture period. Antibiotics such as kanamycin and penicillin may be added to a medium if necessary during culture.

As a result of culture under the above conditions, pyruvic acid is secreted from a strain and then accumulated in the medium.

After completion of culture, pyruvic acid accumulated within the system can be isolated and collected by a conventional method. For example, pyruvic acid is collected by a method that involves separating and removing cells from the culture solution, and then condensing and crystallizing it as pyruvate or a method that uses an ion exchange resin. Moreover, a method that involves performing acid-ether extraction to form phenylhydrazone, followed by precipitation and isolation can also be employed herein.

Specific culture conditions are as described below, but are merely examples. Culture conditions are not limited to these conditions and a person skilled in the art can adequately change the conditions and then perform culture.

### Culture Conditions

(1) Medium composition:

(i) Alginic acid medium: 2-10 (w/v) % sodium alginate, 0.1% ammonium sulfate, 0.1% monopotassium phosphate, 0.1% disodium phosphate, 0.01% yeast extract, 0.01% magnesium sulfate 7 hydrate

(ii) Alginate oligosaccharide medium: 5-15% alginate oligosaccharide, 0.1% ammonium sulfate, 0.1% monopotassium phosphate, 0.1% disodium phosphate, 0.01% yeast extract, 0.01% magnesium sulfate 7 hydrate

(2) pH: pH 6.0-9.0

(3) Culture temperature: 28° C.-37° C.

(4) Culture method: Shake culture

(5) Shaking frequency: Left to stand—145 times of shaking/minute

(6) Culture time: 1 to 3 days

(7) Culture scale: Cultured in a scale of 100 ml within a container such as a 300-ml Erlenmeyer flask, or cultured in a scale of 20 ml within a container such as a 100-ml Erlenmeyer flask.

Biomass at the initiation of culture is represented by a value for A₆₀₀ ranging from 0.05 to 2.0, preferably ranging from 0.075 to 1.25, and is further preferably 0.1 in terms of turbidity. The concentration of alginic acid at the initiation of culture preferably ranges from 2 to 10 (w/v) %, further preferably ranges from 3 to 8 (w/v) %, further preferably ranges from 4 to 7 (w/v) %, and particularly preferably ranges from 4 to 5 (w/v) %.

When culture is performed under the above conditions, alginic acid is consumed after the initiation of culture, and then the production of pyruvic acid begins. The production of pyruvic acid reaches the maximum level within 2 days of culture and increases to day 3 of culture. After day 4 of culture, the assimilation of pyruvic acid begins, and thus the pyruvic acid concentration in the culture supernatant decreases. The oxygen concentration in the medium decreases immediately after the initiation of culture because of oxygen consumption by the strain. However, oxygen is dissolved in the medium because of shake culture, and thus the oxygen concentration starts to increase after 1 day of culture. On day 2 of culture, the dissolved oxygen concentration reaches about 20%, and then reaches saturation.

Regarding the pyruvic acid production (the amount of pyruvic acid produced) when culture is performed under the above conditions, the concentration of pyruvic acid secreted in the medium is a maximum of 2.0 g/l or greater, preferably 2.5 g/l or greater, further preferably 3.0 g/l or greater, and particularly preferably 4.0 g/l or greater. The pyruvic acid production can be achieved preferably on day 2 of culture. Also, through adjustment of the pH of a medium to preferably pH6.0 to 8.0, further preferably pH6.5 to 7.5, further preferably pH6.5 to 7.2, further preferably pH6.5 to 7.0, and particularly preferably pH7.0, pyruvic acid production can further be improved, such that the concentration of pyruvic acid to be secreted in the medium is 5.0 g/l or greater, further preferably 5.5 g/l or greater, and particularly preferably 6.0 g/l or greater. The pyruvic acid production can be preferably achieved on days 2 to 3 of culture.

Pyruvic acid can also be produced by immobilizing the Sphingomonas sp. A1 strain having a disrupted lactate dehydrogenase (LDH) gene and then performing an enzymatic reaction. Examples of a method for immobilizing microorganisms include an entrapment method, a crosslinking method, and a carrier binding method. Examples of an immobilization carrier to be used for immobilization include glass beads, silica gel, polyurethane, polyacrylamide, polyvinyl alcohol, carrageenan, alginic acid, agar, and gelatin.

### Examples

The present invention is hereafter described in greater detail with reference to the following examples, however, the present invention is not limited thereto.

### Example 1

### Production of Pyruvic Acid by Lactate-Dehydrogenase-Deficient Sphingomonas Sp. A1 Strain (LDH Strain)

### Method

A wild-type Sphingomonas sp. A1 strain (WT strain), a lactate-dehydrogenase-gene-deficient Sphingomonas sp. A1 strain (ldh strain), an ethanol-producing Sphingomonas sp. A1 ldh strain (MK3353 strain: prepared by introducing an alcohol dehydrogenase (ADH) gene and a pyruvate decarboxylase (PDC) gene into the ldh strain via pKS13), a control Sphingomonas sp. A1 ldh strain (MK3567 strain: prepared by introducing pKS13 into the ldh strain) were used. In the following examples, in cases in which the MK3353 strain and the MK3567 strain were used, these strains are specified by their strain names (MK3353 strain and MK3567 strain) in order to distinguish between them and strains having no plasmid (i.e., the WT strain and the ldh strain). As a medium, the alginic acid medium described below was used.

The composition of the alginic acid medium is as follows: 0.1 (w/v) % (NH₄)₂SO₄, 0.1 (w/v) % KH₂PO₄, 0.1 (w/v) % Na₂HPO₄, 0.01 (w/v) % MgSO₄.7H₂O, 0.01 (w/v) % yeast extract, and sodium alginate (pH 8.0). As sodium alginate, brown alga-derived sodium alginate having a molecular weight of 300 kDa and an M:G ratio of 3:1 (NACALAI TESQUE, INC.) was used. MK3353 and MK3567 were cultured by adding 20 mg/l tetracycline and 25 mg/l kanamycin, and the lactate-dehydrogenase-gene-deficient strains (ldh strains) were cultured by adding 25 mg/l kanamycin alone. Furthermore, solid culture was performed by adding 1.5% agar to 0.5 (w/v) % alginic acid medium. Cells of the A1 strains grown by solid culture were inoculated in alginic acid media containing 0.8 (w/v) % alginic acid, and then cultured with shaking at 145 strokes per min (spm) at 30° C. for 24 hours, so as to perform preculture. Shake culture was performed using Personal Lt-10F (Taitec). For main culture, cells pre-cultured for about 24 hours were inoculated so that the initial turbidity (OD₆₀₀) was 0.1. Main culture was performed at 30° C. and a shaking frequency of 50, 95, or 145 spm. Main culture of the MK3353 strain and the MK3567 strain was performed using a 300-ml Erlenmeyer flask containing 100 ml of a culture solution and 5% (w/v) alginic acid. The WT strain and the ldh strains were cultured using a 200 ml Erlenmeyer flask containing 20 ml of medium and 0.8%, 2%, 3%, 4% or 5% (w/v) alginic acid. After culture, centrifugation was performed at 20,000 G and 4° C. for 5 minutes to obtain supernatants, and then analysis was conducted. Pyruvic acid was determined using a pyruvic acid determination kit (Roche). Metabolome analysis was conducted by Human Metabolome Technologies using capillary electrophoresis time-of-flight mass spectroscopy (CE-TOFMS).

Dissolved oxygen concentration in a medium was measured using a Fibox3 oxygen sensor (Presens) and an oxygen sensor spot (Presens) provided on the bottom of the Erlenmeyer flask. Relative oxygen concentration in a medium with a saturation of 100% was determined to be 20%. The dissolved oxygen concentration at this time was about 7.5 mg/l.

The quantities of the thus produced ethanol and pyruvic acid were measured by an ethanol assay kit and a pyruvic acid assay kit (Roche Diagnostics). Alginic acid concentration was measured by the carbazole-sulfuric acid method.

Moreover, products in culture supernatants were analyzed by HPLC using Aminex HPX-87H (300×7.8 mm, Bio-Rad) and a RID-10A detector. Furthermore, culture solutions were analyzed by TLC (thin-layer chromatography) using TLC glass plate silica gel 60F₂₅₄ (Merck).

### Results

Metabolome analysis of the supernatants of the culture solutions of the ethanol-producing lactate-dehydrogenase-gene-deficient Sphingomonas sp. A1 strain (ldh strain; MK3353 strain) and the control Sphingomonas sp. A1 ldh strain (MK3567 strain)

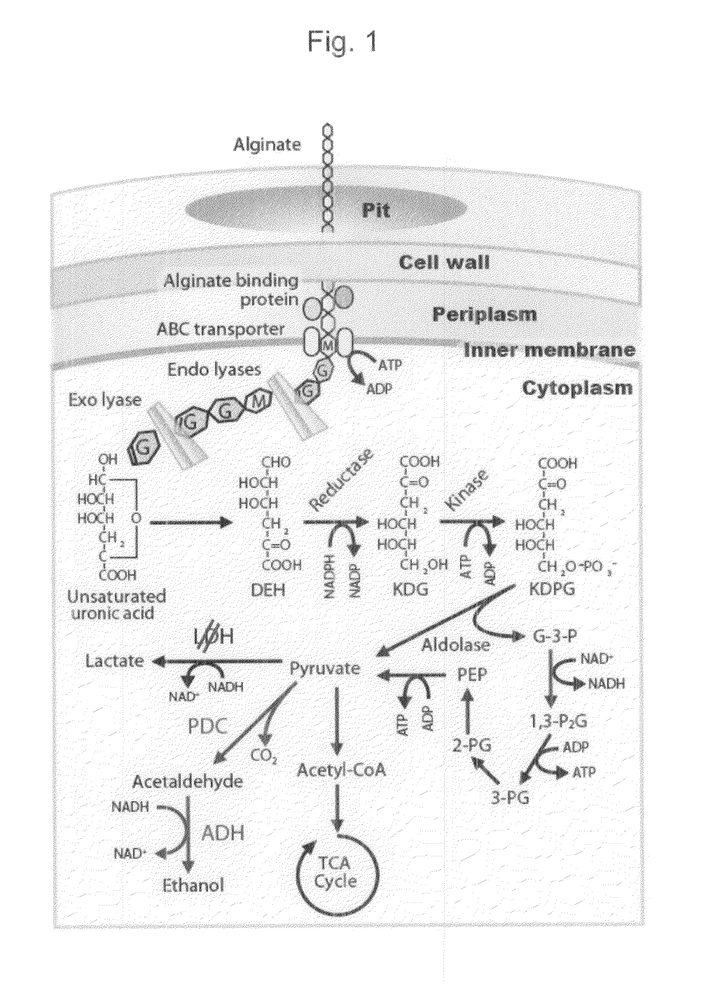

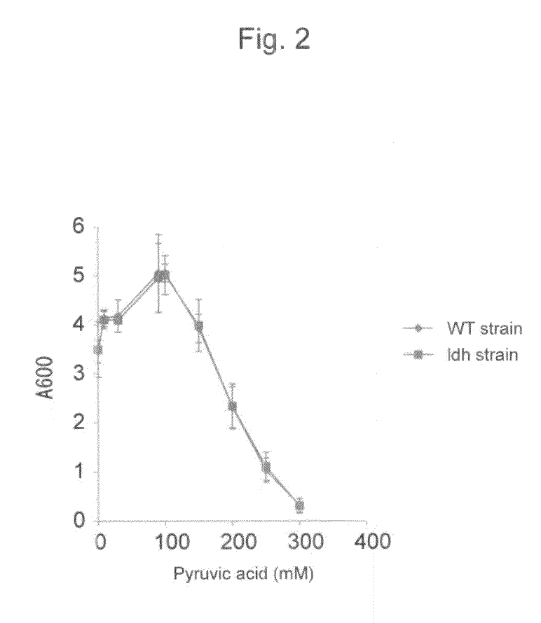

Main culture of the ethanol-producing ldh strain (MK3353 strain) and the control ldh strain (MK3567 strain) was performed at 95 spm for 1, 2, or 4 days. Components contained in the culture supernatants were subjected to metabolome analysis. Among the 61 compounds detected, the concentrations of pyruvic acid and 2-oxoglutaric acid contained in the supernatants of the culture solutions of the MK3567 strain were each higher than 10 mM. Specifically, the concentrations of pyruvic acid contained in the supernatants of the culture solutions of the MK3567 strain were 1.2, 20.2, and 26.2 mM (0.11, 1.78, and 2.31 g/l), respectively, as a result of 1 day, 2 days, and 4 days of culture. The concentrations of 2-oxoglutaric acid contained in the supernatants of the culture solutions of the MK3567 strain were 0.09, 1.6, and 14.4 mM (0.01, 0.23, and 2.10 g/l), respectively. Pyruvic acid was an intermediary metabolite resulting from alginic acid metabolism (FIG. 1); however, the secretion of pyruvic acid into the media was unexpected. The results indicated that pyruvic acid can be produced by the lactate-dehydrogenase-gene-deficient strain. In addition, neither the growth of the Sphingomonas sp. A1 ldh strain nor the growth of the Sphingomonas sp. A1 WT strain was inhibited in the presence of a maximum of 150 mM (13.2 g/l) pyruvic acid (FIG. 2). It was expected that the production of pyruvic acid having at least such concentration (150 mM) is possible via alteration of metabolism or the like. Also, the ethanol-producing MK3353 strain did not secrete pyruvic acid in the medium (data omitted). It was inferred that the pyruvic acid had been consumed by ethanol production due to the action of PDC and ADH (FIG. 1).

### Shaking Frequency of Shake Culture and Pyruvic Acid Production (Amount of Pyruvic Acid Produced)

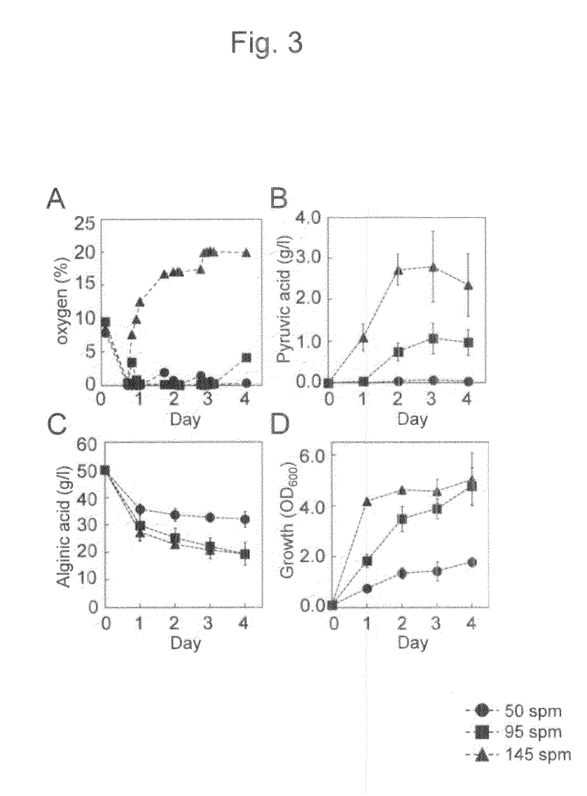

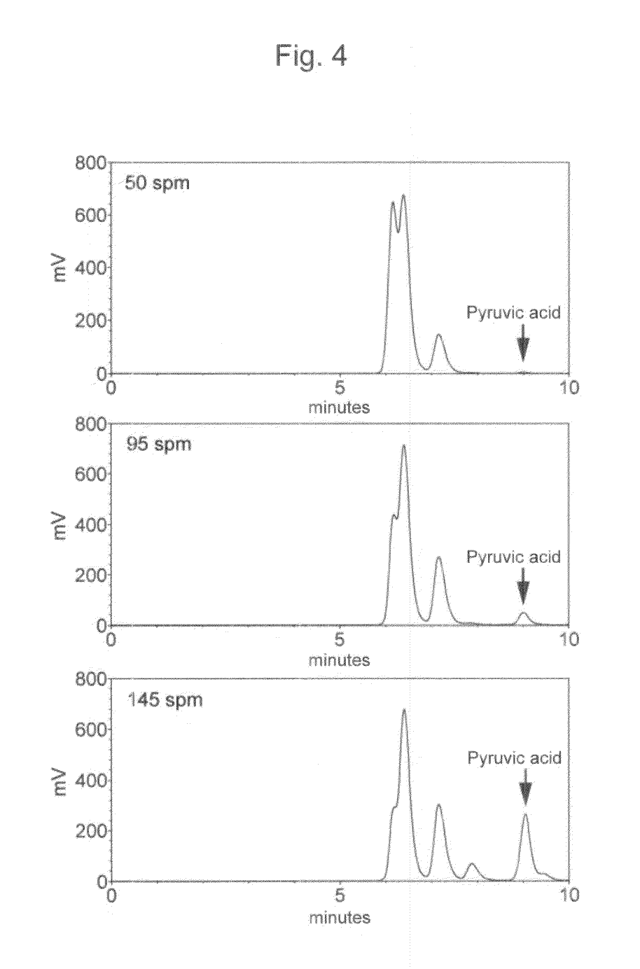

The control Sphingomonas sp. A1 ldh strain (MK3567 strain) was cultured with shaking at 50, 95, or 145 spm for 4 days in a 300-ml Erlenmeyer flask containing 100 ml of 5 (w/v) % alginic acid-containing medium, and then oxygen concentration, pyruvic acid concentration, alginic acid concentration, and the growth of the strain were measured. Aeration was performed to a sufficient extent by culture at 145 spm, aeration was performed to a moderate extent by culture at 95 spm, and aeration was performed to a minor extent by culture at 50 spm. At 50 spm and 95 spm, oxygen concentration was maintained at the lowest level during culture. With culture at 145 spm, oxygen concentration increased to 20% saturating concentration (FIG. 3A). FIGS. 3B, 3C, and 3D show the pyruvic acid production, the amount of alginic acid, and the growth of the strain, respectively. The maximum pyruvic acid concentration was obtained in the case of 145 spm. Pyruvic acid concentration in the case of 95 spm was lower than that in the case of 145 spm, and no pyruvic acid was produced in the case of 50 spm. The growth of the strain correlated with the concentration of produced pyruvic acid. Alginic acid consumption (the amount of alginic acid consumed) was the lowest in the case of culture at 50 spm. Alginic acid consumption in the case of 95 spm was almost the same as that in the case of 145 spm. When the supernatants of the culture solutions obtained after 4 days of culture were analyzed by HPLC, pyruvic acid concentrations in the cases of culture at 145 spm, 95 spm, and 50 spm were 3.33, 0.49, and 0 g/l, respectively. 2-oxoglutaric acid concentrations in the cases of culture at 145 spm, 95 spm, and 50 spm were 0.87, 0.14, and 0 g/l, respectively, which were significantly lower figures than those for pyruvic acid concentration (FIG. 4).

### Initiation Alginic Acid Concentration and Pyruvic Acid Production

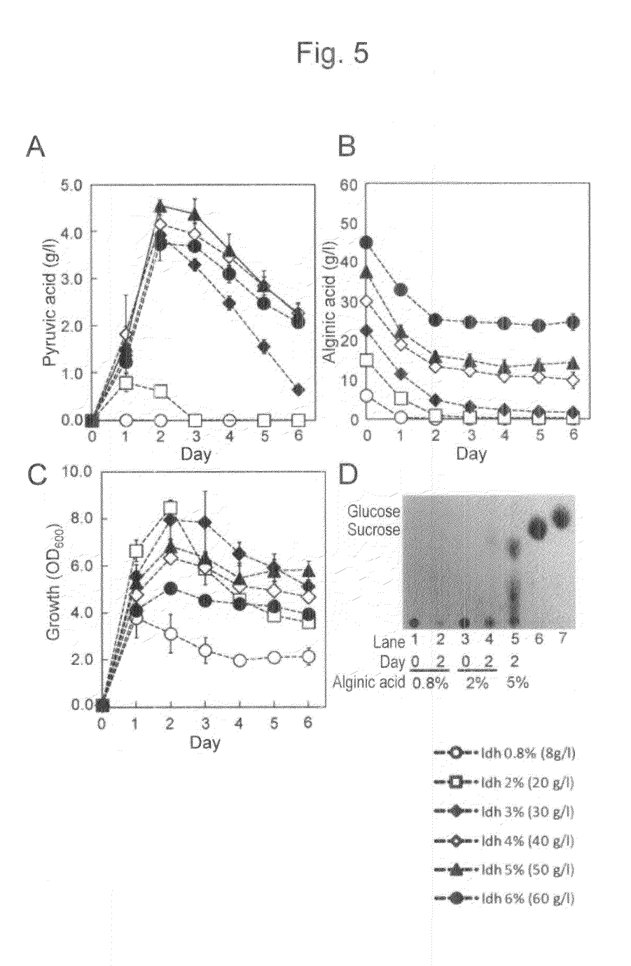

The lactate-dehydrogenase-deficient Sphingomonas sp. A1 strain (ldh strain) was cultured with shaking at 145 spm in a 100-ml Erlenmeyer flask containing 20 ml of a medium containing 0.8, 2, 3, 4, or 5 (w/v) % alginic acid for 6 days. The pyruvic acid concentration, the alginic acid concentration, and the growth of the strain were measured (FIG. 5). FIGS. 5A, 5B, and 5C show pyruvic acid production, the amount of alginic acid, and the growth of the strain. Productivity (mg/l/h), pyruvic acid production (g/g) in relation to alginic acid consumption, and theoretical yield were calculated from the thus obtained data. Theoretical yield was determined to be 100% when 100 g of pyruvic acid was produced from 100 g of alginic acid. The calculation results are shown in Table 1.

As shown in Table 1, pyruvic acid production was low at the initiation alginic acid concentration of 0.8 (w/v) %; however, pyruvic acid concentration (g/l) and productivity (mg/l/h) were maximum at the initiation alginic acid concentration of 5 (w/v) %. Pyruvic acid production (g/g) in relation to alginic acid consumption and theoretical yield were maximum at the initiation alginic acid concentration of 4 (w/v) %. The growth rate on day 2 was high at the initiation alginic acid concentrations of 2 and 3 (w/v) %. FIG. 5D shows the results of TLC (thin-layer chromatography) for culture supernatants.

TABLE 1

| Initiation | Alginic | Pyruvic | | Pyruvic acid | Theo- |
|---|---|---|---|---|---|
| alginic | acid | acid | Produc- | produced/Alginic | retical |
| acid | consumed | produced | tivity | acid consumed | yield |
| (g/l) | (g/l) | (g/l) | (mg/l/h) | (g/g) | (%) ^{d} |
| 8 | 7.3 ^{b} | 0.01 ^{b} | 0.42 | 0.001 | 0.10 |
| 20 | 12.9 ^{b} | 0.79 ^{b} | 32.9 | 0.06 | 6.1 |
| 30 | 23.5 ^{c} | 3.93 ^{c} | 81.7 | 0.17 | 16.7 |
| 40 | 22.3 ^{c} | 4.16 ^{c} | 86.7 | 0.19 | 18.6 |
| 50 | 28.7 ^{c} | 4.56 ^{c} | 95.0 | 0.16 | 15.9 |
| 60 | 26.4 ^{c} | 3.74 ^{c} | 77.9 | 0.14 | 14.2 |
| 50 (WT) ^{e} | 29.5 ^{c} | 3.21 ^{c} | 67.1 | 0.11 | 10.9 |

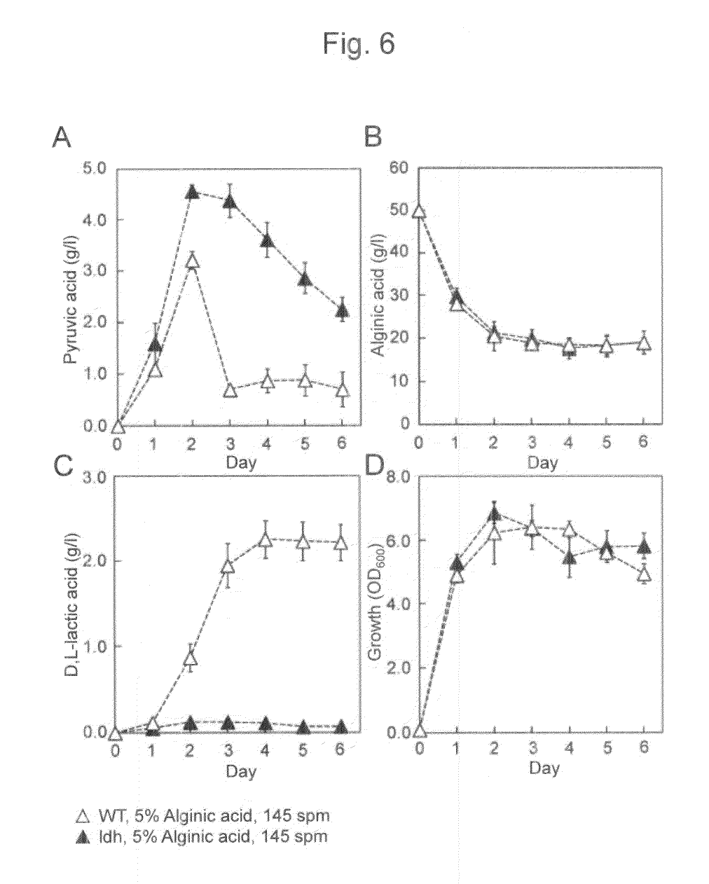

^{a} Calculated from the data in FIG. 5 and FIG. 6.
^{b} Concentrations of alginic acid consumed and pyruvic acid produced after 1 day of culture
^{c} Concentrations of alginic acid consumed and pyruvic acid produced after 2 days of culture
^{d} Theoretical yield was determined to be 100% when 100 g of pyruvic acid was produced from 100 g of alginic acid consumed.
^{e} Results for the wild type strain (WT).

### Confirmation of the Effects of the Disruption of the Lactate Dehydrogenase (LDH) Gene on Pyruvic Acid Concentration

The wild-type Sphingomonas sp. A1 strain and the lactate-dehydrogenase-deficient Sphingomonas sp. A1 strain (ldh strain) were cultured with shaking at 145 spm using 5 (w/v) % alginic acid medium, and they were then compared in terms of pyruvic acid production. The results are shown in FIG. 6. FIGS. 6A, 6B, 6C, and 6D show pyruvic acid production, the amount of alginic acid, the amount of lactic acid, and the growth of the strains. Alginic acid consumption and growth figures were almost the same for both strains; however, more pyruvic acid was produced with the ldh strain than with the wild-type strain. Moreover, the pyruvic acid concentration in the wild-type strain decreased sharply. The wild-type strain produced D-lactic acid; however, the ldh strain produced almost no D-lactic acid (and neither the wild-type strain nor the ldh strain produced L-lactic acid). The results indicated that LDH gene deficiency accelerates the production of pyruvic acid.

Assimilation of Pyruvic Acid by the Sphingomonas Sp. A1 Strain

As shown in FIG. 3B, FIG. 5A, and FIG. 6A, pyruvic acid concentrations decreased sharply after the concentrations had reached their maximum values. This was significant particularly in the wild-type strain. The results indicate that Sphingomonas sp. A1 uses pyruvic acid as a carbon source, clearly confirming with certainty that the wild-type Sphingomonas sp. A1 strain uses pyruvic acid as a carbon source. Specifically, it was suggested that the Sphingomonas sp. A1 strain uses alginic acid, secretes pyruvic acid, and recycles secreted pyruvic acid after it stops using alginic acid.

### Example 2

### Initial Alginic Acid Concentration and Pyruvic Acid Production (2-10 (w/v) % Alginic Acid)

With an initial alginic acid concentration of 2-10 (w/v) %, the lactate-dehydrogenase-deficient Sphingomonas sp. A1 strain (ldh strain) was cultured for 6 days by a method similar to that described in example 1.

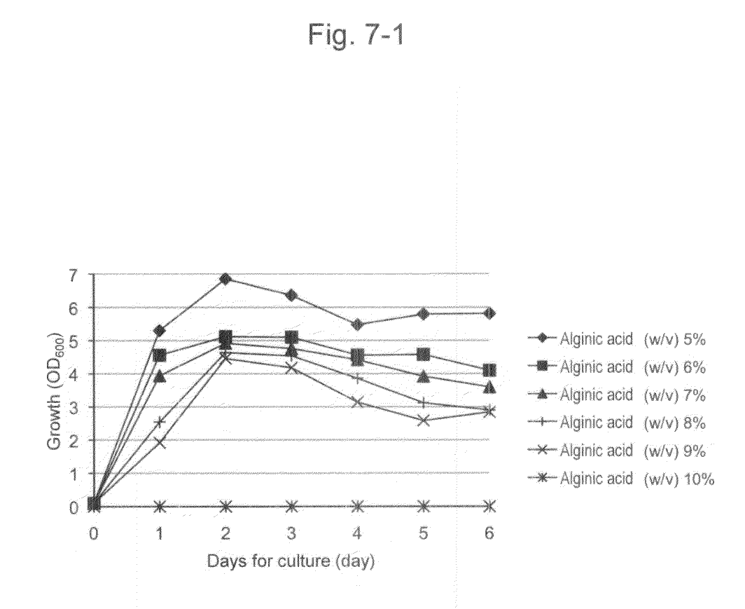

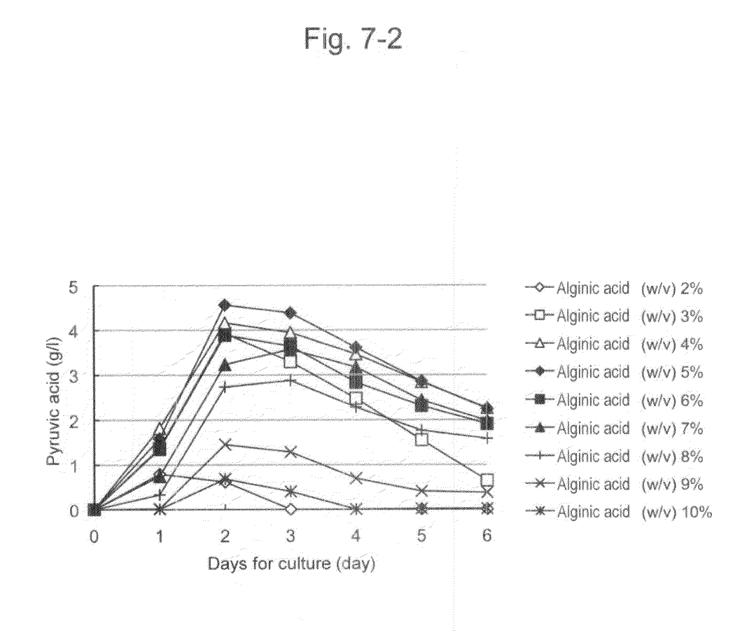

FIG. 7-1 shows the growth of the lactate-dehydrogenase-deficient Sphingomonas sp. A1 strain (ldh strain) when the initial alginic acid concentration ranged from 5% to 10%. FIG. 7-2 shows the amount of pyruvic acid produced when the initial alginic acid concentration ranged from 2% to 10%. As shown in FIG. 7-1, the growth of the strain reached a maximum when the initial alginic acid concentration was 5%. However, once the concentration exceeded 5%, the growth rate decreased as the initial alginic acid concentration increased. Also, as shown in FIG. 7-2, the amount of pyruvic acid produced reached a maximum when the initial alginic acid concentration was 5%, and decreased regardless of whether the concentration was lower or higher than 5%. When the initial alginic acid concentration was 2%, 9% or 10%, pyruvic acid production was significantly low. Moreover, both the growth of the strain and pyruvic acid production reached a maximum within 2 to 3 days of culture.

### Example 3

### Effects of pH on Pyruvic Acid Production by Lactate-Dehydrogenase-Deficient Sphingomonas Sp. A1 Strain (A1ΔLDH) (MK2651 Strain)

### Method

### Strain and Medium

In this example, the lactate-dehydrogenase-gene-deficient ldh strain (designated as MK2651 strain) prepared from the A1 bacterial strain of the genus Sphingomonas used in examples 1 and 2 was used. Alginic acid medium was used to culture the strain. The composition of the alginic acid medium (pH7.9) was composed of 0.5, 0.8 or 5% (w/v) sodium alginate [derived from Eisenia bicyclis, average molecular weight of 110 kDa, (Nacalai Tesque, Kyoto, Japan)], 0.1% (w/v) (NH₄)₂SO₄, 0.1% (w/v) KH₂PO₄, 0.1% (w/v) Na₂HPO₄, 0.01% (w/v) MgSO₄.7H₂O, and 0.01% (w/v) yeast extract (Nacalai Tesque). In addition, hereinafter, alginic acid media containing 0.5, 0.8, and 5.0% (w/v) sodium alginate are denoted as 0.5, 0.8, and 5.0% alginic acid media, respectively. In the case of solid medium, agar (Nacalai Tesque) was added to 1.5% (w/v). Each alginic acid medium was also supplemented with kanamycin (Km) (Wako Pure Chemical Industries, Osaka, Japan) to a final concentration of 25 μg/ml.

### Culture and pH Adjustment

Preculture was performed by inoculating the MK2651 strain that had been previously cultured (pre-preculture) for 2 to 3 days on a 0.5% alginic acid solid medium, into 3 ml of a 0.8% alginic acid medium. This was then cultured with a shaking frequency of 145 strokes per min (spm) and 30° C. for about 24 hours. Main culture was performed by adding the pre-cultured MK2651 strain to 20 ml (200-ml Erlenmeyer flask) of a 5% alginic acid medium so that the initial turbidity (A₆₀₀) was 0.1, and then culturing the strain with shaking at 145 spm and 30° C. After the initiation of the main culture, the pH of the culture solution was adjusted every 24 hours to 5.0, 6.0, 6.5, 7.0, 7.5 or 8.0. pH was measured using pH meter F-21 (Horiba, Kyoto, Japan) and adjusted using 5N HCl and 5N NaOH. Moreover, sampling of each culture solution was performed before pH adjustment. In addition, unless mentioned otherwise, pH measurement and pH adjustment for each culture solution were performed by transferring the total volume of the culture solution to a 50-ml falcon tube. Thereafter, the culture solution was transferred back to a 200-ml Erlenmeyer flask and then culture was continued. In addition, pH measurement in the control experiment was performed by the following two methods. In one method, pH was measured by dipping a pH meter into a culture solution that had been kept within a 200-ml Erlenmeyer flask without being transferred to a falcon tube. In the other method, a culture solution was transferred to a falcon tube, however, a pH meter was not dipped into the culture solution and the pH of the culture solution was not measured.

### Pyruvic Acid Determination

Each culture solution sampled was subjected to centrifugation (4° C., 20,000 g, 5 minutes) and then pyruvic acid concentration in the thus obtained supernatant solution was measured using an F-kit (Roche Diagnostics). In addition, a calibration curve was created every measurement.

### Results

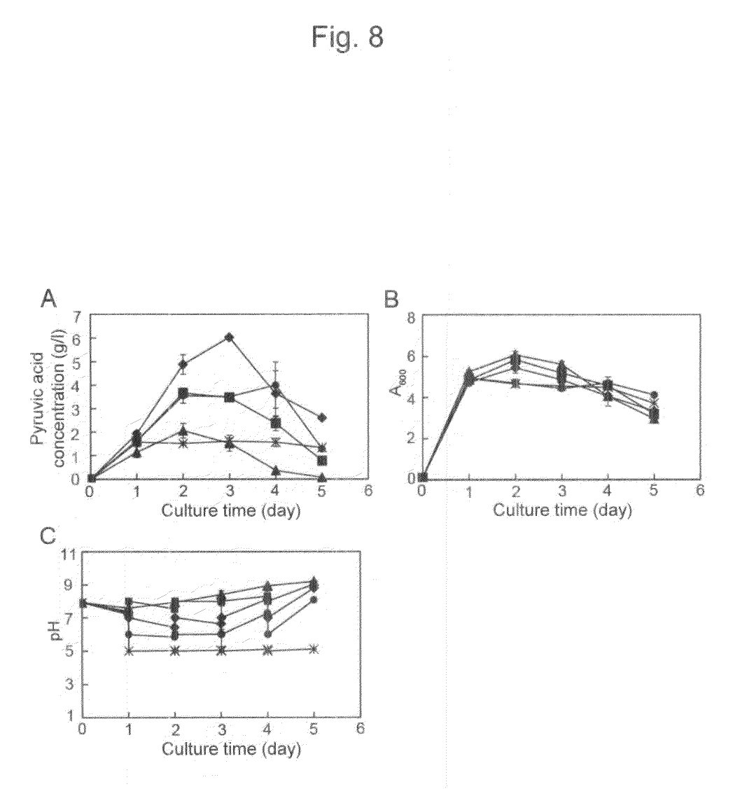

Culture of the MK2651 strain was initiated in a 5% alginic acid medium (pH7.9), and then the pH of the culture solution was adjusted every 24 hours to 5.0, 6.0, 7.0, or 8.0. Culture was continued while controlling the pH. Also, as a control for these solutions, the MK2651 strain was cultured with pH measurement taking place only every 24 hours after the transfer of the culture solution to a falcon tube. The results are shown in FIG. 8. FIG. 8, (A) shows pyruvic acid concentration, (B) shows turbidity (A₆₀₀), and (C) shows pH of the culture solution of the MK2651 strain. In FIG. 8, * denotes pH5.0, • denotes the results in the case of pH6.0, ♦ denotes the results in the case of pH7.0, and ▪ denotes the results in the case of pH8.0. Furthermore, ▴ denotes the results of culturing without pH adjustment (the total amount of the culture solution was transferred to a falcon tube, pH was measured, and then the solution was transferred back to an Erlenmeyer flask). The following average values, maximum values and minimum values resulting from “n” times of culture are shown in this figure (n=2 for day 1 to day 4 of culture, and n=1 for day 5 of culture). In addition, average values of measured pHs before pH adjustment of each culture solution (measured value when n=1) were: day 1 (▴: 7.58, ▪: 7.39, •: 7.32, ♦: 7.21, ▪: 7.35); day 2 (▴: 7.94, ▪: 5.03, •: 5.85, ♦: 6.41, ▪: 7.53); day 3 (▴: 8.40, ▪: 5.05, •: 6.02, ♦: 6.63, ▪: 7.99); day 4 (▴: 8.94, ▪: 5.10, •: 7.27, ♦: 8.12, ▪: 8.31); and day 5 (▴: 9.20, ▪: 5.11, •: 8.07, ♦: 8.80, ▪: 9.04). As shown in FIG. 8, a maximum of 6.0 g/l pyruvic acid was obtained on day 3 of culture with the system wherein pH adjustment to 7.0 was performed every 24 hours.

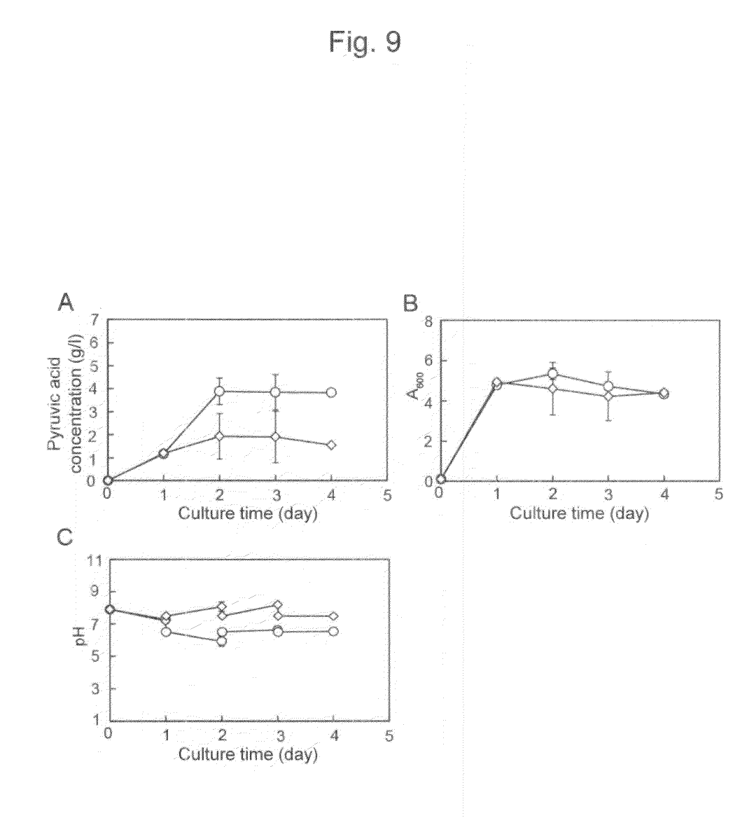

Next, the pH of the culture solution was adjusted to 6.5 or 7.5 every 24 hours after the initiation of the culture of the MK2651 strain, to examine whether these pH values were more suitable than pH 7.0 for the production of pyruvic acid. The results are shown in FIG. 9. FIG. 9 (A) shows pyruvic acid concentration, (B) shows turbidity (A₆₀₀), and (C) shows pH of the culture solution of the MK2651 strain. In FIG. 9, ∘ denotes the results in the case of pH6.5, and ⋄ denotes the results in the case of 047.5. The following average values, maximum values, and minimum values resulting from “n” times of culture are shown in this figure: (n=2 for day 1 to day 3 of culture, and n=1 for day 4 of culture). In addition, average values (measured values when n=1) of measured pH values before pH adjustment of each culture solution were: day 1 (∘: 7.30, ⋄: 7.20), day 2 (∘: 5.93, ⋄: 8.08), day 3 (∘: 6.63, ⋄: 8.20), and day 4 (∘: 6.53, ⋄: 7.49). As shown in FIG. 9, the pyruvic acid concentration in the culture solution obtained by adjusting pH to pH6.5 or 7.5 every 24 hours was lower than that of the culture solution in the system wherein pH adjustment to pH7.0 had been performed. Therefore, this suggested that high concentrations of pyruvic acid can be obtained by adjusting the culture solution of the MK2651 strain to pH7.0 every 24 hours upon the flask-scale production of pyruvic acid by the MK2651 strain.

### INDUSTRIAL APPLICABILITY

According to the method of the present invention, pyruvic acid can be produced from the polysaccharide, alginic acid, of brown algae.

All publications, patents, and patent applications cited herein are incorporated herein by reference in their entirety.

What is claimed is:

1. A method for producing pyruvic acid, comprising culturing a lactate-dehydrogenase-gene-deficient Sphingomonas sp. A1 strain (ldh strain) in a medium containing alginic acid and causing the strain to produce pyruvic acid from alginic acid and then to secrete pyruvic acid into the medium.

2. The method for producing pyruvic acid according to claim 1, wherein the concentration of alginic acid at the initiation of culture ranges from 2 to 10 (w/v) % and the biomass of the lactate-dehydrogenase-gene-deficient Sphingomonas sp. A1 strain (ldh strain) at the initiation of culture is represented by a value for A₆₀₀ ranging from 0.075 to 1.25 in terms of turbidity.

3. The method for producing pyruvic acid according to claim 1, wherein culture is performed under conditions in which the maximum dissolved oxygen concentration in a medium is 15% or more.

4. The method for producing pyruvic acid according to claim 3, wherein culture is performed for 2 to 3 days.

5. The method for producing pyruvic acid according to claim 4, wherein culture is performed by shake culture at a shaking frequency of 100 spm (strokes per minutes) or higher.

6. The method for producing pyruvic acid according to claim 5, wherein culture is performed at pH 6.0 to 8.0.

7. The method for producing pyruvic acid according to claim 6, wherein the concentration of pyruvic acid secreted into the medium on day 2 of culture is 2.5 g/l or greater.

8. The method for producing pyruvic acid according to claim 7, wherein the concentration of pyruvic acid secreted into the medium on days 2 to 3 of culture is 5 g/l or greater.


SEQUENCE LISTING (8 sequences)

SEQ ID NO: 1 - DNA, 975 bp, Sphingomonas sp.
atgacaagcgccaccaaggcacagagcataccgctgcccgcagcgacgctcgacgtgcgttcgaaaccgctcgaacgcctgtggaaagacatcaaacgcgactggctgttgtatgccatgttgctacccaccatcatctggttcctgatctttctgtacaagccgatgatcggcctgcagatggcgttcaagcagtacagcgcctggaaaggcatcgccggcagtccgtggatcgggttcgaccacttcgtcacgctgttccagagcgaacagttcattcgcgccatcaagaacacactgaccctgtcgggtctgtcgctgttgttcggcttcccgatgccgatcctgctggcgctgatgatcaacgaggtctactcgaagggttatcgcaaggcagtgcagaccatcgtctatctgcctcacttcatctccatcgtgatcgtggccggcctcgtggtgacattcctgtcgcccagcaccggcgtggtcaacaacatgctgagctggatcggtctggaccgggtgtacttcctgacccagccggagtggttccgtccgatctacatcagctcgaacatctggaaggaagcgggcttcgattcgatcgtgtatctggccgcgatcatgagcatcaacccggcgctgtatgaatcggcacaggtggacggtgccacgcgctggcagatgatcacgcgcatcaccctgccctgcatcgtcccgaccatcgcggtgctgctggtgatccgcctgggccacatcctggaagtgggcttcgagtacatcatcctgctgtaccagccgacgacctacgaaacggccgacgtgatcagtacctacatctatcgtctgggcctgcaaggggcgcgctacgacatcgctaccgccgccggcattttcaatgccgtggtggccctggtgatcgtgctgttcgccaaccacatgagccgccgtatcaccaagaccggcgtgttctga

SEQ ID NO: 2 - DNA, 882 bp, Sphingomonas sp.
atgctcgccacacctttctatagccgctccgacaggatcttcggcatcgtcaacgccgtcctgctgggcatcttcgcgctctgtgcgctgtacccgatcatctacatcttctcgatgtcgatcagctcgggcgcggcggtcacacagggccgcgtattcctgcttccggtggacatcgacttttcggcctacggccgcgtgctccacgacaagctgttctggacgtcgtatgccaacaccattttctacacggtgttcggtgtggtcacgagcctgatcttcatcgtgcccggtgcgtatgcgctgtccaagccgcgtatccgtggccgccgcgtgttcggcttcatcatcgccttcacgatgtggttcaacgcagggatgatcccgttcttcctgaacatgcgcgacctcgggctgctcgacaaccgcttcggcatcttgatcggcttcgcctgtaacgcgttcaacatcatcctgatgcgcaactacttcgaatcgatctcagcatcgttcgaagaggccgcacgcatggatggcgccaatgacctgcagattctgtggaaggtgtacatcccgctggccaagccggcgctggccaccatcaccctgctctgcgccatctctcgctggaacggttacttctgggccatggtgctgctgcgcgccgaagagaagatcccgctgcaggtgtacttgaagaagaccatcgtcgatctgaacgtcaacgaagagttcgcaggcgccctgctcaccaactcgtattcgatggaaaccgtggtgggcgccatcatcgtgatgtcgatcatccccgtgatcatcgtctacccggtggtgcagaagtacttcaccaagggtgtgatgctcggaggcgtaaaagaataa

SEQ ID NO: 3 - DNA, 1092 bp, Sphingomonas sp.
gtggtagcaagcgtcagcattcagaacgtcgtcaagcgttacgacaagacgactgtggtccatggtgtcagcctggatatcgaaccgggtgaattcgtcgtgctggtcggcccttcgggctgcggcaagtcgacgacactacgcatggtggccggtctggaagaaatcagcggcggcacgatcaggatcgacggccgcgtcatcaacgacctcgcgccaaaagatcgcgacgtcgccatggtgttccagaactacgcgctctacccgcacctgaatgtgcgcgacaacatctcctttggcctgcgcctgaagcgcaccaagaaatccgtcatcgatgcagccgtgaagacggccgccgacatcctcggcctgcaaccgctgctcgaacgcaagccttccgatctttccggcggtcagcgtcaacgcgttgccatgggccgcgcgatcgtgcgcgacccgaaggtcttcctcttcgacgaaccgctctccaacctcgacgccaaactgcgcacgcagatgcgcgccgaaatcaagcggctgcaccaacgcctgggcaccaccgtcatttatgtgacgcacgatcaggtcgaagccatgacattggcagatcgcatcgtggtcatgcgcgacggtctgatcgaacagatcggcaagccgatggacctgttcctccatccggccaataccttcgtggccagcttcattggttcgccgccgatgaacctgatgccggcacgcattgccgtcgacagcacgcagcacgttgaattgaacggtggcaaccgcatcagcctgctgccccgtgcaggcacacatctggcaccgggtcaggaagtggtgtttggcattcgcccggaagacgtcacgctcgacggcgtcgaaggctccgaacgtgcgcagatcaaggccacggtggacatcgtcgaaccgctcggttcggaatcgatcctccacgctacggtcggcgatcactcgctggtggtgaaggtgggcggcctcaatgaagtacatccgggcgatccggtcacgctgcatgtcgatctgacacgcgtgcacctgttcgacgcccagtcacaggcctctatttactga

SEQ ID NO: 4 - DNA, 1581 bp, Sphingomonas sp.
atgaaaaagctggtactggccatggccatcggaagttccgcattgatgttcgcggctccgtcggtggtcgcgcgcgaggcgacgtgggtgaccgaaaagccactcacgctcaagatccacatgcacttccgcgacaagtgggtgtgggacgagaactggccggtggccagagaagtcgcccgcctcaccaacgtcaagctcgtcggtgtagccaacagggccgctaccaacagccaggaacagttcaacctgatgatggcctcgggccagttgccggacatcgtcggcggcgacaacctgaaggacaagttcatccgctacgggatggaaggcgcgttcattccgctcaacaagttgatcgatcagaacgcccccaacctgaaggcattcttcaagacacaccccgaagtccagcgcgccatcacagcgccggacggcaatatctattacctgccctacgtgccggacgggctggtgtcgcgcgggtacttcatccgccaggactggctcgacaaactgcatctgaagaccccgcagaccgtcgatgagctgtacaccgtgctcaaggcgttcaaggaaaaagacccgaacggcaacggcaaggccgatgagattccgttcatcaaccgcgacccggaagaggtcttccgtctcgtgaatttctggggcgctcgctcgaccggttcgaatacgtggatggatttctacgtcgagaacggcaagatcaagcatccgttcgccgaagtcgccttcaaggacggcatcaagcacgtggcccagtggtacaaggaaggtctcatcgaccccgagatcttcacgcgcaaggcgcgctcacgcgaacagaccttcggcaacaacatcggcggcatgacgcatgactggttcgccagcacggcgctgttcaacgatgcgctgtccaagaacatccccggcttcaagctcgtgccgatggcgccgcccatcaacagcaagggccagcgttgggaagaagacgctcgccagattccgcgccccgacggctgggcgatcacggcgaccaacaagaacccggtcgaaaccatcaagctgttcgacttctacttcgggccgaagggccgcgagctgtccaacttcggcgtaccgggcctgacgtatgacatcaagaacggcaagccggtctacaaggacacggtgctgaaggccgcccagccggtgaacaaccagatgtacgacatcggcgcgcagattcccatcggcttctggcaggactacgagtacgagcgtcaatggaccaacgacgtcgcactccagggcatcgacatgtacatcaagaacaagtacgtgctgccccaattcaccggcgtcaacctgactgtcgaagagcgcgagatctacgacaaatactggccggacgtgaagacctacatgttcgaaatgggccagtcctgggtgatgggcaccaaggacccggaaaaaacgtggaacgactatcagcagcaactgaagaaccgcggtttctatcaggtcatgatcgttatgcagaaggcctatgaccggcagtacggcggggcagcgaagccagcccaggtcggagccaagtaa

SEQ ID NO: 5 - DNA, 1551 bp, Sphingomonas sp.
atgaagaagatgatgttgtccgtcgcagcagtggccacgctgatggcctttgccgcgcccgtcgccacggcgaaggaggccacctgggtgaccgacaagcccttgacgctgaagatccatatgcacttccgcgacaagtgggtgtgggacgagaactggccggtcgccaaggaatcgttccgcctgaccaacgtgaagctgcaaagcgtcgccaacaaggccgccacgaacagccaggaacagttcaacctgatgatggcttcgggcgacctgccggacgtcgttggcggtgacaacctgaaggacaagttcattcagtacgggcaggaaggcgcattcgttccgctcaacaagctgatcgaccaatacgccccgcatatcaaggcgttcttcaaatcgcacccggaagtcgagcgcgccatcaaggcgccggacggcaacatctacttcattccttacgtgccggacggcgtggtcgcgcgcggctacttcatccgcgaagactggctcaagaagctcaacctgaagccgcctcagaacatcgatgaactctacaccgtgctcaaggcgttcaaggaaaaagacccgaacggcaacggcaaggccgatgaagtgcccttcatcgaccgccatccggacgaagtcttccgcctggtgaacttctggggcgcgcgttcgtcggggtccgataactacatggacttctacatcgacaacggccgcgtcaagcacccgtgggccgaaaccgccttccgcgacggcatgaagcatgtggcccagtggtacaaggaaggcctgatcgacaaggaaatcttcacccgcaaggcacgcgcacgcgaacaaatgttcggtggcaatctgggcggcttcacgcacgactggttcgccagcacgatgaccttcaacgaaggtctggccaagacggtgccgggcttcaagctgattccgatcgccccgccgaccaacagcaagggccagcgctgggaagaagactcccgccagaaggttcgtcccgatggctgggccatcacggtgaagaacaagaacccggtggaaaccatcaagttctttgacttctacttcagccgtccgggccgcgacatctccaactttggcgttccgggtgtcacgtatgacatcaagaacggcaaggcggtgttcaaggattccgtgctcaagtcgccgcagccggtcaacaaccagctctatgacatgggcgcacagatccccatcggcttctggcaggactacgactacgaacgtcagtggaccacgcctgaagcgcaggccggcatcgacatgtacgtcaagggcaagtacgtcatgcctggctttgaaggcgtgaacatgacgcgtgaagagcgcgccatctacgacaagtactgggccgacgttcgcacgtacatgtatgaaatgggccaagcctgggtgatgggcaccaaggacgtggacaagacctgggacgagtaccagcgtcagctcaagctgcgcggtctctaccaagtgctgcagatgatgcagcaagcctacgaccgtcaatacaagaactaa

SEQ ID NO: 6 - DNA, 1926 bp, Sphingomonas sp.
atgcctttggcatgccttgcaacgacgcgcgtgggggctgcgcgcgaaaaatctggagacagttcgatgtttgacatcccgttcccggggcatggccgtcgtctggccgtcgccgcgcttgcgttcgcaggttgtgccttcgcgggcagcctgcaggcacaccccttcgaccaggccgtcgtgaaagaccccacggcctcgtatgtcgacgtcaaggcgcgtcgtaccttcttgcagagcgggcagctcgatgaccgcctcaaggcagcgctgcccaaggagtacgactgcacgaccgaggcaacgcctaacccgcagcaaggcgagatggtcattccgcgccgttatctttccggcaatcacggcccggtgaatccggactacgaaccggtggtgacgctttatcgtgatttcgagaaaatttccgccacgcttggaaatctctacgttgcgacgggcaagccggtgtacgccacttgtctgctgaacatgctggacaagtgggccaaggccgacgcgttgctcaactacgaccccaagtcgcagtcgtggtaccaagtcgaatggtcggcggcgaccgccgcctttgccctgtcgacgatgatggccgagccgaacgtcgacacagcccagcgcgagcgtgtggtgaagtggctcaaccgtgtggcgcgccatcagacgagctttccggggggcgacacgagttgctgcaacaatcactcgtattggcgcggtcaggaagcgaccatcatcggcgtgatcagcaaggacgatgaactcttccgttgggggctgggccgttatgtgcaggcgatggggctgatcaatgaagacggcagcttcgtgcatgaaatgacgcgtcacgaacagtccttgcactatcagaactacgccatgctgccgctgacgatgattgccgaaacggcatcacgtcagggtatcgatctgtacgcgtacaaagagaacggtcgcgatattcactcagcgcgcaagtttgtctttgctgcggtgaagaacccggacctcatcaagaagtacgcttccgagccgcaggatacgcgtgccttcaagccggggcggggcgacctgaactggatcgaataccagcgcgcgcgctttggttttgccgatgagctgggcttcatgacggtgccgatcttcgatccgcgtaccggtggttcgggcactttgctggcgtacaaaccgcaaggcgcggcagcgcaggcgccggtgtccgccccggcagcggcgcattcgtcgatcgacctgtcgaagtggaagctgcagattccggtcgacccgatcgatgtcgccacgcgtgatctgctcaagggttatcaggacaagtacttctacgtcgacaaggatggctcgcttgccttctggtgtccggccagcggtttcaagacgacggccaacacgaagtacccgcgcagcgagctgcgcgaaatgctcgatcccgacaaccacgcggtgaattggggctggcaaggcacccacgaaatgaatctgcgcggagcggtgatgcacgtgtcgcccagtggcaagaccattgtcatgcagattcatgccgtcatgcccgatggctcgaatgcgccgccgctggtcaaggggcagttctacaagaacacgctggattttcttgtgaagaactcggccgctggcggcaaggacacgcactacgtgttcgaaggcatcgaactgggcaagccgtacgatgcgcagatcaaagtggtggacggtgtgttgtcgatgacggtcaacgggcagaccaagaccgtcgatttcgtcgccaaggacgccggctggaaagacctgaagttctatttcaaggcgggcaattacttgcaggaccgccaggctgacgggtcggacaccagcgcgctggtcaagttgtacaagctcgacgtaaagcactcgagctga

SEQ ID NO: 7 - DNA, 2289 bp, Sphingomonas sp.
gtgatgaagaagctggaacagccgcagtccgcggacatgacaatcagatatttccccgacagtgacacgctgaccgaaaatccgccgcgctttctgtggctgccggaactcgacagccaggcgcgctacgccgtgcgcgtggtgtcggacgcggggaaagagacggtgttcggcgacatccagcacaacttcttccgccccgacgtcacactgacgccgggtagctacacctggtcttacgctctgtgggacgcagccgcaggcaaggtcgactccgactggagctccccgcgcagcttcgttctgcccgccgacgcgcccgctgtgcccggcatgcgccataaagaacggtacgccgagtgcgacatgacgcacccgcgcctgtggctgtcgcaaagcgaagccaccgcgttgggcaagagcgtcgccaccgacccggatcactgcaagtgggccaacttcatggccaagtcggtcacgccatggatgtcgcgcgagatcatcgccgagccgcagccctaccccaacaacaagcgcacgccggccctgtggcgcaagatgtacatcgactgccaggaagtgatctacgcgatccgccacctggccatcgccggtcgtgtgctgggtgatgcggccatgaccgcccgcgccaaggaatggctgctcgccgtcgccgcgtgggacgtgcgtggcccgacctgccgcgactacaacgacgaagccgccttccgcatcgcggcagcactggcctggggctacgactggctgcacgacgaactgtcggacatcgaacgcgccaacgtgcgcgaatcgctgacggtgcgcacgcgcgaagtcgccaaccacgtgatcaaccgcgccaagattcatatcttcccgtacgacagccacgccgtgcgttcgatttcgtcggtgctggtgccctgctccatcgtgctgctgggcgaagtgcccgaagcccagaagtggctcgactactccatcgatttctacgacgcgatctacccaccgtggggcggcgtggacggcggctgggccgagggtccgcactactggatgacggccatggcctacttcaccgaggcggccaacctcgtgttgaagtacttcaagcatgacctgtacaagcgcccgttcttccagagcactggctggttcccgctctacaccaaggccccggacacgcgtcgtgcgtgctttggcgatgactccacgctcggcgacctgccctgcctgaaggtgggctacaacctgcgccaattcgccggcgtcaccggcaacccgtacttccagtggtacttcgaagaggtgtgccgcaacgatccgggcaccgaaatggagttctacaactacggttggtgggacctgaacttcgacgacgtgcagtacctgcacgacttcccgcacatcgaagcgaaggcgccgtccgacatcgacaccgtcaagtactttggcgatgttggctgggtcgcgctgcaacggcacatggacaagccggatcagcacatccagttcctcaccaaggccagcgacttcggctcgatcagtcacagccatggagaccagggtgcgttcctgctgtatggctacggcgaagacctggccatccagagcggctactacatcggtttcggcaccaccatgcacaaggaatggcgccgactgaccaaatcgaagaacgccattctcatcgacgggaaggggcaatattcaggttcgaacaaagccgaatgcctcaaggcgcaaggtcatgtgctggaagtaggcacgcgtaacggcgcgcacttcatcagcctcgacccgaccgacgcgtacaaggccgaggtgccctacctcacccgctatcgtcgcgacattcacttcgtgcatgaccgcttcttcgtcatcgtcgacgacgtcgaactggaacaggacggcagcgtgcagtggctcatgcacaccttcaagccgtgcgaactcggctcgcaggtgttccgctaccacggtgacaaagcgggcctgaatggcgaattcgtctattgctcgtccggcccggtgagcatcgagaacacggtctcgtacgccaatatcgatcagagcgaagttgaaggactgccaccgcaatccacgctggtggcgagcaccggcgccgcacgtcgccacacattggtgaccttgctcacaccgtacgcactgtcggcgccgctgcgcgtattccacttcaacgacgatcagggcttctcgaccaacctgtacttccaggacggcaacaacgagatgtacaccatcacgttgcccaaaccgttctaa

SEQ ID NO: 8 - DNA, 843 bp, Sphingomonas sp.
atgacatccatcgccgtcacctcaggccccggccgttttcacacaccgataacgagagacatatccatgttcccagacctcaaaggcaagcgcgttctgatcaccggttcgtcgcagggcattggccttgccaccgcccgcctcttcgcccgtgcaggcgccaaggtcggcctgcacggccgcaaggcacccgccaacatcgatgagaccatcgccagcatgcgcgccgacggcggcgatgccgctttcttcgcggcggacctggccacctccgaagcctgccagcaactggtcgatgagttcgtggccaagttcggtggcatcgacgtgctcatcaacaatgctggcggcctggtcggccgcaagccgctccctgagatcgacgatacgttctacgacgccgtcatggacgccaacatccgctcggtcgtaatgaccaccaagttcgcgctgccccatctggcggccgcagccaaggcatccggccagaccagcgcggtgatcagcaccgggtcgatcgctggccacaccggtggcggccccggcgcaggtctgtatggcgcagccaaggctttcctgcacaacgtgcacaagaactgggtcgacttccacaccaaggacggcgtgcggttcaacattgtgtcgccgggtaccgtcgacaccgccttccatgcagacaagacgcaagacgtgcgcgaccgtatctccaacggcatcccgatggggcgcttcggcaccgccgaagaaatggcgccggcattcttgttcttcgcctcgcacctggccagcggttacatcaccggtcaggtgctggacatcaacggcgggcagtacaagcactga